(19) United States
(12) Patent Application Publication
(10) Pub. No.: US 2008/0194567 A1
(43) Pub. Date: Aug. 14, 2008

(54) Combination Treatment for Enhancing Diuresis

(76) Inventors: Piero Pollesello, Grankulla (FI); Jyrki Lilleberg, Helsinki (FI)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/908,403

(22) PCT Filed: Mar. 14, 2006
(86) PCT No.: PCT/FI06/00087

§ 371 Date: Oct. 11, 2007

Related U.S. Application Data
(60) Provisional application No. 60/661,012, filed on Mar. 14, 2005.

(51) Int. Cl. A61K 31/50

(52) U.S. Cl. 514/247

(57) ABSTRACT

A combination therapy for promoting diuresis in a patient comprises administering levosimendan or its active metabolite or any of their pharmaceutically acceptable salts in conjunction with a diuretic to a patient. The combination therapy provides diuretic effect also in patients who are refractory to standard diuretic therapy.

### TECHNICAL FIELD

The present invention relates to a method of treatment of patients using a levosimendan compound or a pharmaceutically acceptable salt thereof in combination with a diuretic agent. The invention also relates to a medical product comprising a levosimendan compound or a pharmaceutically acceptable salt thereof and a diuretic as a combined preparation.

### BACKGROUND OF THE INVENTION

Levosimendan, which is the (−)-enantiomer of [[4-(1,4,5,6-tetrahydro-4-methyl-6-oxo-3-pyridazinyl)phenyl]hydrazono]propanedinitrile, is currently used for the short term treatment of patients who suffer from acutely decompensated severe heart failure. The drug increases contractile force of the heart myocardium by enhancing the sensitivity of myofilaments to calcium. Levosimendan and a method for its preparation are described in U.S. Pat. No. 5,569,657.

Diuresis is generally defined as an increase in the rate of urine formation. Drugs producing such effect are generally referred to as diuretics and the site of their action is generally the kidney. Diuretics are commonly used for example in the treatment of hypertension and in the management of various conditions of fluid overload and oedema such as pulmonary vascular congestion. Such conditions may be associated with e.g. liver diseases, corticosteroid therapy or cardiovascular disorders such as congestive heart failure. A significant problem encountered with diuretic therapy is that adequate diuretic effect is not always achieved or the patients may become refractory to the diuretic treatment, i.e. the patients begin to respond less and less to the medication until they do not respond at all.

### SUMMARY OF THE INVENTION

It has now been found that administration of a pharmaceutically effective amount of a levosimendan compound or a pharmaceutically acceptable salt thereof together with a pharmaceutically effective amount of a diuretic provides synergistic diuretic effect. Therefore, the combination is particularly useful for the treatment of patients suffering from fluid overload. The combination was found to be effective also when sufficient diuresis can not be obtained by a diuretic therapy alone, e.g. in patients who are refractory to standard diuretic therapy.

Thus, in one aspect the present invention provides a method of promoting diuresis in a patient comprising administering to said patient a levosimendan compound or a pharmaceutically acceptable salt thereof in conjunction with a diuretic.

In another aspect the invention provides a medical product comprising, separately or together, as active ingredients a levosimendan compound or a pharmaceutically acceptable salt thereof and a diuretic as a combined preparation.

In another aspect invention provides a pharmaceutical composition comprising as active ingredients a levosimendan compound or a pharmaceutically acceptable salt thereof and a diuretic.

In another aspect the invention provides the use of a levosimendan compound or a pharmaceutically acceptable salt thereof in conjunction with a diuretic in the manufacture of a medicament for promoting diuresis in a patient.

In another aspect the invention provides the use of a levosimendan compound or a pharmaceutically acceptable salt thereof in conjunction with a diuretic in the manufacture of a combined preparation for simultaneous, separate or sequential administration.

In still another aspect the invention provides the use of a levosimendan compound or a pharmaceutically acceptable salt thereof in the manufacture of a medicament for promoting diuresis in a patient.

### DETAILED DESCRIPTION OF THE INVENTION

The method of the invention relates to a combination therapy for promoting diuresis in a patient who suffer from fluid overload, particularly when sufficient diuresis can not be obtained by diuretic therapy alone, by administering to a patient in need thereof a levosimendan compound or a pharmaceutically acceptable salt thereof in conjunction with a diuretic agent. The method and combination of the invention is particularly useful in the treatment of hypertension and in the management of various conditions of fluid overload and oedema. Such conditions may be associated with e.g. liver diseases, corticosteroid therapy or cardiovascular disorders such as congestive heart failure. In a certain embodiments, the patient being treated by the methods of the present invention suffers from renal impairment. In other embodiments, the patient does not suffer from renal impairment. These patients include those who suffer from congestive heart failure, or other diseases that result in fluid overload, without having yet disrupted kidney function. In some embodiments, the patient being treated by the methods of the present invention is refractory to standard diuretic therapy. In other embodiments, the patient is not refractory to standard diuretic therapy.

The active ingredients may be administered simultaneously, separately or sequentially. In particular, the method comprises administering to a patient an amount of active ingredients or combination thereof which is effective to promote or induce diuresis in the patient. Preferably, the method comprises administering to a patient a synergistically effective amount of the combination. The administration routes of the active ingredients include, but are not limited to, enteral, e.g. oral or rectal, or parenteral, e.g. intravenous, intramuscular, intraperitoneal or transdermal.

As used herein, the term “levosimendan compound” refers to any racemic mixture or enantiomer of levosimendan or a racemic mixture or enantiomer of the active metabolite of levosimendan. The term “levosimendan” specifically refers to the (−)-enantiomer of [4-(1,4,5,6-tetrahydro-4-methyl-6-oxo-3-pyridazinyl)phenyl]-hydrazono]propanedinitrile. The term also is intended to encompass combinations of levosimendan and its active metabolite. The active metabolite of levosimendan is particularly (R)—N-[4-(1,4,5,6-tetrahydro-4-methyl-6-oxo-3-pyridazinyl)phenyl]-acetamide (II).

The term “patient” means animals, preferably mammals, and humans.

The effective amount of a levosimendan compound to be administered to a subject depends upon the condition to be treated, the route of administration, age, weight and the condition of the patient. Levosimendan or its active metabolite (II) is generally administered orally to man in dose from about 0.01 to 10 mg, preferably from about 0.1 to 5 mg, given once a day or divided into several doses a day.

Levosimendan can be administered by intravenous infusion using the infusion rate from about 0.01 to 5 μg/kg/min, typically from about 0.02 to 3 μg/kg/min, for example from about 0.05 to 0.4 μg/kg/min.

Among the diuretics useful in the combination medicament of the invention are thiazide and related sulfonamide diuretics hydrochlorothiazide, bendroflumethiazide, benzthiazide, chlorothiazide, chlorthalidone, cyclothiazide, hydroflumethiazide, indapamide, methyclothiazide, metolazone, polythiazide, altizide, clopamide, bemetizide, quinethazone and trichlormethiazide; potassium sparing diuretics and aldosterone receptor antagonists such as amiloride, eplerenone, spironolactone, potassium canrenoate and triamterene; loop diuretics such as bumetanide, torsemide, ethacrynic acid, azosemide, piretanide and furosemide; carbonic anhydrase inhibitors such as acetazolamide, dorzolamide, dichlorphenamide and methazolamide; ORL-1 agonists such as ZP120; and pharmaceutically acceptable salts thereof; and combinations thereof. Particularly preferred are furosemide, amiloride hydrochloride, spironolactone and hydrochlorothiazide.

According to the invention, a diuretic may be administered in daily doses, which are clinically accepted for such agents. For example, a suitable daily dose for furosemide is about 20-40 mg, for amiloride hydrochloride about 5-20 mg, for spironolactone about 50-400 mg, and for hydrochlorothiazide about 10-100 mg, depending upon the condition to be treated, the route of administration, age, weight and the condition of the patient.

The combination may be supplemented with one or more other active ingredients.

The active ingredients or the combination thereof may be administered daily or several times a day, or periodically depending on the patient's needs.

The active ingredients can be formulated into pharmaceutical dosage forms suitable for the treatment according to the present invention using the principles known in the art. They are given to a patient as such or preferably in combination with suitable pharmaceutical excipients in the form of tablets, granules, capsules, suppositories, emulsions, suspensions or solutions whereby the contents of the active compound in the formulation is from about 0.5 to 100% per weight. Choosing suitable ingredients for the composition is a routine for those of ordinary skill in the art. It is evident that suitable carriers, solvents, gel forming ingredients, dispersion forming ingredients, antioxidants, colours, sweeteners, wetting compounds, release controlling components and other ingredients normally used in this field of technology may be also used.

The active ingredients may be formulated in the same pharmaceutical formulation. Alternatively, the active ingredients are formulated as separate pharmaceutical dosage forms. The combination of the pharmaceutical dosage forms may be packaged as a single medical product or kit for use in the method of the invention, optionally together with a package insert instructing to the correct use of the medical product.

For example, according to one embodiment of the invention, the invention provides a medical kit comprising a first pharmaceutical dosage form comprising a levosimendan compound or a pharmaceutically acceptable salt thereof a second pharmaceutical dosage form comprising a diuretic, a package for containing said first and second dosage forms, and optionally instructions for simultaneous, separate or sequential administration of said first and second dosage forms to a patient.

Formulations suitable for intravenous administration such as injection or infusion formulation, comprise sterile isotonic solutions of the active ingredient and vehicle, preferably aqueous solutions. Typically an intravenous infusion solution of a levosimendan compound comprises from about 0.01 to 0.1 mg/ml of a levosimendan compound. Typical intravenous infusion solution for e.g. furosemide comprises about 0.5-1 mg/ml of furosemide. The pharmaceutical formulation may be also in the form of an intravenous infusion concentrate to be diluted with an aqueous vehicle before use. Such concentrate may comprise as a vehicle a pharmaceutically acceptable organic solvent such as dehydrated ethanol.

For oral administration of the active ingredients in tablet form, suitable carriers and excipients include e.g. lactose, corn starch, magnesium stearate, calcium phosphate and tale. For oral administration in capsule form, useful carriers and excipients include e.g. lactose, corn starch, magnesium stearate and talc. For controlled release oral compositions release controlling components can be used. Typical release controlling components include hydrophilic gel forming polymers such as hydroxypropylmethyl cellulose, hydroxypropyl cellulose, carboxymethyl celluloses, alginic acid or a mixture thereof vegetable fats and oils including vegetable solid oils such as hydrogenated soybean oil, hardened castor oil or castor seed oil (sold under trade name Cutina HR), cotton seed oil (sold under the trade names Sterotex or Lubritab) or a mixture thereof; fatty acid esters such as triglycerides of saturated fatty acids or their mixtures e.g. glyceryl tristearates, glyceryl tripalmitates, glyceryl trimyristates, glyceryl tribehenates (sold under the trade name Compritol) and glyceryl palmitostearic acid ester.

Tablets can be prepared by mixing the active ingredient with the carriers and excipients and compressing the powdery mixture into tablets. Capsules can be prepared by mixing the active ingredient with the carriers and excipients and placing the powdery mixture in capsules, e.g. hard gelatin capsules. Typically a tablet or a capsule comprises from about 0.1 to 10 mg, more typically 0.2 to 5 mg, of a levosimendan compound or/and from about 5 to 50 mg of hydrochlorothiazide, from about 20 to 40 mg of furosemide, from about 20 to 100 mg of spironolactone or from about 2 to 5 mg of amiloride hydrochloride.

The diuretic may be included in the levosimendan formulation or may be formulated separately as described above using principles well known in the art.

Salts of levosimendan may be prepared by known methods. Pharmaceutically acceptable salts are useful as active medicaments, however, preferred salts are the salts with alkali or alkaline earth metals.

### EXAMPLES

### Pharmaceutical Examples

### Example 1

### Concentrate Solution for Intravenous Infusion

| | | | |
|---|---|---|---|
| (a) | levosimendan | 2.5 | mg/ml |
| (b) | Kollidon PF12 | 10 | mg/ml |
| (c) | citric acid | 2 | mg/ml |
| (d) | dehydrated ethanol | ad 1 | ml (785 mg) |

The concentrate solution was prepared by dissolving citric acid, Kollidon PF121 and levosimendan to dehydrated ethanol in the sterilized preparation vessel under stirring. The resulting bulk solution was filtered through a sterile filter (0.22 μm). The sterile filtered bulk solution was then aseptically filled into 8 ml and 10 ml injection vials (with 5 ml and 10 ml filling volumes) and closed with rubber closures.

The concentrate solution for intravenous infusion is diluted with an aqueous vehicle before use. Typically the concentrate solution is diluted with aqueous isotonic vehicles, such as 5% glucose solution or 0.9% NaCl solution so as to obtain an aqueous intravenous solution, wherein the amount of levosimendan is generally within the range of about 0.001-1.0 mg/ml, preferably about 0.01-0.1 mg/ml.

### Example 2

Hard gelatin capsule size 3

| | |
|---|---|
| Levosimendan | 2.0 mg |
| Lactose | 198 mg |

The pharmaceutical preparation in the form of a capsule was prepared by mixing levosimendan with lactose and placing the powdery mixture in hard gelatin capsule.

### Example 3

Hard gelatin capsule size 3

| | |
|---|---|
| Furosemide | 40.0 mg |
| Lactose | 198 mg |

### Example 4

Hard gelatin capsule size 3

| | |
|---|---|
| Hydrochlorothiazide | 25.0 mg |
| Levosimendan | 1.0 mg |
| Lactose | 199 mg |

### Experiments

### Experiment 1

Sixty-six years old man was admitted to hospital due to congestive heart failure. In cardiac echocardiography he had ejection fraction below 30%, moderate regurgitation of mitral valve and dilative cardiomyopathy. According to coronary angiography invasive procedures such as angioplasty or coronary by-pass surgery were not possible. During the first four days at hospital he received diuretics (furosemide and spironolactone) as well as lisinopril, bisoprolol, antibiotics and allopurinol without any positive response. His dyspnea worsened and peripheral oedema increased. Levosimendan infusion was added with the dose of 0.05 microg/kg/min and was increased to 0.1 microg/kg/min after two hours, the infusion lasted 24 hours, no bolus infusion was used due to low blood pressure. During the next day patient told that he felt better and dyspnea and periphera oedema decreased. His urinary volumes increased markedly. The daily urine volumes varied between 850 and 1850 ml before the start of levosimendan, and increased up to 6350 ml on the day he received levosimendan, and were 3800 ml on the day after levosimendan infusion, after that they went down to normal.

### Experiment 2

Seventy-two years old man was admitted to hospital due to severely worsened dyspnea. He suffered from coronary artery disease with no possibilities for re-vascularisation. He had ejection fraction below 35%, markedly elevated end-diastolic pressure and moderate mitral regurgitation. Lung oedema was seen in his chest-X-ray and slight increase in troponin I value. He was treated with positive pressure ventilation, vasodilators and diuretics for three days without positive response. Levosimendan fusion with a bolus and continuous infusion rate up to 0.2 microg/kg/min was added. The infusion lasted 24 hours. Before the infusion his daily urinary volumes varied between 2150 and 2730 ml. On the day levosimendan was started his urinary volume was 2650 and increased to 3855, 2890 and 2790 ml during the next consecutive days, and his fluid balance was 8500 ml negative during the four next days after the infusion of levosimendan.

### Experiment 3

Seventy-nine years old man was admitted to hospital due to worsened dyspnea. He suffered from idiopathic dilating cardiomyopathy and his left ventricular ejection fraction was 26%. He was treated with diuretics (furosemide and spironolactone) as well as perindopril, bisoprolol and digitalis without positive response for one week. Levosimendan infusion of 0.05 microg/kg/min was added. During the next 24 hours he urinated 3500 ml and during the next five days his weight decreased from 66.5 kg to 62.5 kg, although it had not changed at all before the infusion of levosimendan.

1. A medical product comprising, separately or together, as active ingredients a levosimendan compound or a pharmaceutically acceptable salt thereof and a diuretic as a combined preparation.

2. A pharmaceutical composition comprising as active ingredients a levosimendan compound or a pharmaceutically acceptable salt thereof and a diuretic.

3. A method of promoting diuresis in a patient comprising administering to said patient a levosimendan compound or a pharmaceutically acceptable salt thereof in conjunction with a diuretic.

4. A method of claim 3, wherein the patient is refractory to standard diuretic therapy.

5. Use of a levosimendan compound or a pharmaceutically acceptable salt thereof in conjunction with a diuretic in the manufacture of a medicament for promoting diuresis in a patient.

6. Use of a levosimendan compound or a pharmaceutically acceptable salt thereof in conjunction with a diuretic in the manufacture of a combined preparation for simultaneous, separate or sequential administration.

7. Use of a levosimendan compound or a pharmaceutically acceptable salt thereof in the manufacture of a medicament for promoting diuresis in a patient.
